(19) United States
(12) Patent Application Publication
(10) Pub. No.: US 2022/0323405 A1
(43) Pub. Date: Oct. 13, 2022

(54) COMBINATION PRODUCT USING 3,4-METHYLENEDIOXYMETHAMPHETAMINE AND CARVEDILOL FOR THE TREATMENT OF PSYCHIATRIC DISORDERS

(71) Applicant: Universitätsspital Basel, Basel (CH)

(72) Inventor: Matthias Emanuel LIECHTI, Oberwil (CH)

(73) Assignee: Universitätsspital Basel, Basel (CH)

(21) Appl. No.: 17/692,094

(22) Filed: Mar. 10, 2022

Related U.S. Application Data
(60) Provisional application No. 63/172,238, filed on Apr. 8, 2021.

(51) Int. Cl. A61K 31/36; A61P 9/12; A61K 31/403

(52) U.S. Cl. CPC A61K 31/36; A61P 9/12; A61K 31/403

(57) ABSTRACT

A composition for treating an individual while reducing acute adverse effects of an empathogen/entactogen, including effective amounts of the empathogen/entactogen and an adverse effect-reducing agent that blocks adverse effects of the empathogen/entactogen. A method of treating an individual with an empathogen/entactogen and reducing its acute adverse effects, by administering an empathogen/entactogen to the individual, administering an adverse effect-reducing agent to the individual, and reducing the acute adverse effects of the empathogen/entactogen. A method of stopping the acute cardiostimulant and hyperthermic action of an empathogen/entactogen in an individual, by administering an adverse effect-reducing agent to the individual after the individual has taken the empathogen/entactogen and stopping the acute adverse effects of the empathogen/entactogen. A method of treating an individual at risk of cardiovascular events and/or hyperthermia with an empathogen/entactogen.

### GRANT INFORMATION

Research in this application was supported in part by a grant from the Swiss National Science Foundation (Grant No. 323230_126231).

### BACKGROUND OF THE INVENTION

### 1. Technical Field

The present invention relates to compositions and methods for using 3,4-methylenedioxymethamphetamine (MDMA) in combination with carvedilol or any other adrenergic α/β-receptor antagonists in medical treatments. Specifically, the present invention relates to methods and means for reducing the acute cardiovascular stimulant and thermogenic effects of MDAM while preserving its beneficial and therapeutic effects on mood.

### 2. Background Art

MDMA (FIG. 1A) is a psychoactive drug that alters mood and perception and has been investigated as an adjunct in psychotherapy for posttraumatic stress disorder and further studies are proposed for a range of other medical conditions (Luoma et al., 2020; Mithoefer et al., 2019; Mithoefer et al., 2010; Oehen et al., 2013; Sessa et al., 2019).

MDMA acutely induces subjective effects including heightened mood, openness, trust, and enhanced empathy (Hysek et al., 2014; Schmid et al., 2014) that are considered beneficial when MDMA is used to assist psychotherapy.

However, MDMA also has sympathomimetic properties and acutely produces cardiovascular stimulant effects and increases body temperature (Liechti, 2014; Vizeli & Liechti, 2017). These sympathomimetic effects are unwanted when MDMA is used therapeutically and are a medical problem in patients with cardiovascular risk factors or established diseases. In some cases, MDMA can even increase blood pressure or body temperature to levels considered dangerous in healthy subjects (Vizeli & Liechti, 2017; Vollenweider et al., 1998). Additionally, when used recreationally, MDMA is well-known to produce medical problems including fatalities and typically including hyperthermia, tachycardia, increased blood pressure (Green et al., 2004; Hall & Henry, 2006; Liechti et al., 2005; Noseda et al., 2021; Rosenson et al., 2007).

In particular, hyperthermia is a typical complication of MDMA use and fatal cases of multi-organ failure due to hyperpyrexia are regularly reported in the context recreational MDMA use (Liechti, 2014; Mills et al., 2003; Parrott, 2012). No proven effective drug treatment is established to reverse or even prevent hyperthermia.

MDMA releases serotonin, norepinephrine, dopamine, and oxytocin (Hysek et al., 2014; Hysek et al., 2012b). The psychotropic effects of MDMA are thought to mainly depend on the release of serotonin and possibly oxytocin (Dumont et al., 2009; Hysek et al., 2012b; Liechti et al., 2000). The stimulant adverse effects of MDMA on blood pressure and body temperature are mediated by adrenergic α and β-receptors (Hysek et al., 2013a; Hysek et al., 2012a; Hysek et al., 2010; Liechti, 2014; Mills et al., 2004; Sprague et al., 2005; Sprague et al., 2007).

Past preclinical and clinical research showed beneficial effects of carvedilol (FIG. 1B) on MDMA-induced hyperthermia in rodents (Sprague et al., 2005) and reduced the thermogenic effects of MDMA in healthy human subjects (Hysek et al., 2013b).

Specifically, research in mice showed that the thermogenic effects of MDMA involve stimulation of uncoupling protein-3 in mitochondria (Mills et al., 2003). An al-antagonist administered 30 minutes before MDMA significantly attenuated the MDMA-induced increase in rectal temperature but not in skeletal muscle in rats (Sprague et al., 2003). A β3-antagonist administered 30 minutes before MDMA significantly attenuated the increase in skeletal muscle temperature but had no effect on the rise in rectal temperature (Sprague et al., 2003). The combination resulted in an abolishment of MDMA-induced hyperthermia (Sprague et al., 2003) indicating a role for both α1- and β3-receptors in MDMA-induced hyperthermia. It was proposed that norepinephrine release mediated by MDMA creates heat generation through both activation of uncoupling protein (UCP3) along with α1-β3-adrenoreceptor stimulation and loss of heat dissipation through vasoconstriction (Mills et al., 2004; Sprague et al., 2004a).

In rats, MDMA induced norepinephrine levels. Administration of β-blockers before MDMA had no effect on the thermogenic response. In contrast, carvedilol administered before or after MDMA prevented this hyperthermic response. Moreover, when administered 1 hour after MDMA, carvedilol completely reversed established hyperthermia. This preclinical research showed that α1 and β3-adrenergic receptors can contribute to the mediation of MDMA-induced hyperthermia and that drugs targeting these receptors, such as carvedilol, warrant further investigation as novel therapies for the treatment of psychostimulant-induced hyperthermia and its sequelae.

First studies in humans were also conducted. The β receptor blocker pindolol was administered before MDMA and prevented MDMA-induced increases in heart rate. Peak values (mean±SD) for heart rate were 84±13 beats/minute after MDMA vs 69±7 beats/minute after pindolol-MDMA. In contrast, pindolol pretreatment had no effect on increases in mean arterial blood pressure (MAP) after MDMA. Peak MAP values were 115±11 mm Hg after MDMA vs 114±11 mm Hg after pindolol-MDMA. Pindolol did not change adverse effects of MDMA. This study showed that β-blockers can prevent increases in heart rate but not hypertensive and other adverse effects of MDMA.

Another study tested effects of the α1-noradrenergic receptor antagonist doxazosin on the acute response to MDMA in 16 healthy subjects. Doxazosin (8 mg/d) or placebo was administered for 3 days before MDMA (125 mg) or placebo using a randomized, double-blind, placebo-controlled, 4-session, crossover design. Doxazosin reduced MDMA-induced elevations in blood pressure and body temperature, but enhanced tachycardia associated with MDMA. The results indicate that α1-adrenergic receptors contribute to the acute cardiostimulant and thermogenic effects of MDMA in humans.

Another study tested effects of the combined α1- and β-adrenoceptor antagonist carvedilol on the cardiostimulant, thermogenic, and subjective responses to MDMA in 16 healthy subjects. Carvedilol (50 mg) or placebo was administered 1 hour before MDMA (125 mg) or placebo using a randomized, double-blind, placebo-controlled, four-period crossover design. Carvedilol reduced MDMA-induced elevations in blood pressure, heart rate and body temperature. Carvedilol did not affect the subjective effects of MDMA including MDMA-induced good drug effects and drug liking. Carvedilol did not alter the plasma exposure to MDMA. The study documented that α1- and β-adrenoceptors contribute to the cardiostimulant and thermogenic effects of MDMA in humans but not to its psychotropic effects. It was concluded that carvedilol could be useful in the treatment of cardiovascular and hyperthermic complications associated with ecstasy use.

The past research illustrates that carvedilol was mainly perceived as a treatment option in cases of hypertension or hyperthermia. However, the combined use of carvedilol with MDMA to prevent complications associated with MDMA alone was not further conceptualized or proposed. There remains a need for treatment combinations with MDMA that prevent MDMA-induced cardiovascular stimulation and hyperthermia in particular in patients at increased risk for cardiovascular side effects.

### SUMMARY OF THE INVENTION

The present invention provides for a composition for treating an individual while reducing acute adverse effects of an empathogen/entactogen, including effective amounts of the empathogen/entactogen and an adverse effect-reducing agent that blocks adverse effects of the empathogen/entactogen.

The present invention provides for a method of treating an individual with an empathogen/entactogen and reducing its acute adverse effects, by administering an empathogen/entactogen to the individual, administering an adverse effect-reducing agent to the individual, and reducing the acute adverse effects of the empathogen/entactogen.

The present invention also provides for a method of stopping the acute cardiostimulant and hyperthermic action of an empathogen/entactogen in an individual, by administering an adverse effect-reducing agent to the individual after the individual has taken the empathogen/entactogen and stopping the acute adverse effects of the empathogen/entactogen.

The present invention also provides for a method of treating an individual at risk of cardiovascular events and/or hyperthermia with an empathogen/entactogen, by administering an empathogen/entactogen to the individual, administering an adverse effect-reducing agent to the individual, and providing psychotropic effects of empathogen/entactogen to the individual while reducing risk of cardiovascular events and/or hyperthermia.

### DESCRIPTION OF THE DRAWINGS

The advantages of the present invention are readily appreciated as the same becomes better understood by reference to the following detailed description when considered in connection with the accompanying drawings wherein:

FIG. 1A shows the chemical structure of MDMA and FIG. 1B shows the chemical structure of carvedilol;

FIGS. 2A-2F are prior art graphs showing acute effects of MDMA on blood pressure (FIGS. 2A-2B), heart rate (FIG. 2C), rate pressure product (FIG. 2D), body temperature (FIG. 2E), and pupil size (FIG. 2F);

FIG. 3 is a prior art graph showing acute effects of MDMA on body temperature;

FIG. 4 is a prior art schematic of the mechanisms involved with MDMA-induced hyperthermia;

FIG. 5A is a prior art graph showing effects of pretreatment with pindolol on MDMA-induced effects on body temperature, and FIG. 5B is a prior art graph showing peak values;

FIG. 6A is a prior art graph showing effects of pretreatment with pindolol on MDMA-induced effects on heart rate, and FIG. 6B is a prior art graph showing peak values;

FIG. 7A is a prior art graph showing effects of pretreatment with pindolol on MDMA-induced effects on blood pressure, and FIG. 7B is a prior art graph showing peak values;

FIGS. 8A-8C are prior art graphs showing effects of pretreatment with doxazosin on MDMA-induced effects on blood pressure (FIG. 8A), heart rate (FIG. 8B), and body temperature (FIG. 8C);

FIGS. 9A-9D are prior art graphs showing effects of pretreatment with carvedilol on MDMA-induced effects on blood pressure (FIGS. 9A-9B), heart rate (FIG. 9C), and body temperature (FIG. 9D);

FIGS. 10A-10F are prior art graphs showing effects of pretreatment with carvedilol on subjective effects of MDMA of any drug effect (FIG. 10A), good drug effect (FIG. 10B), drug liking (FIG. 10C), happiness (FIG. 10D), openness (FIG. 10E), and closeness (FIG. 10F);

FIGS. 11A-11C are graphs showing effects of MDMA alone, carvedilol alone, carvedilol prior to MDMA and carvedilol co-administered with MDMA at the same time (modeled data) on systolic (FIG. 11A), diastolic (FIG. 11B), and mean arterial blood pressure (FIG. 11C) in healthy humans; and

FIGS. 12A-12B are graphs showing effects of MDMA alone, carvedilol alone, carvedilol prior to MDMA and carvedilol co-administered with MDMA at the same time (modeled data) on heart rate (FIG. 12A) and body temperature (FIG. 12B) in healthy humans.

### DETAILED DESCRIPTION OF THE INVENTION

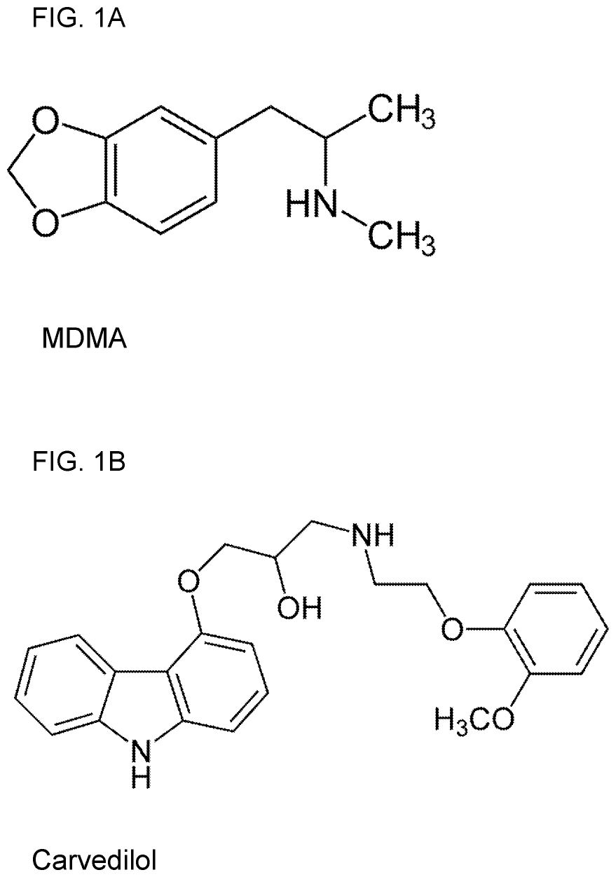

The present invention provides generally for compositions and methods for a safer use of empathogens/entactogens (such as MDMA (FIG. 1A)) for treatment of medical conditions. More specifically, the present invention provides for a composition for treating an individual while reducing adverse acute effects of an empathogen/entactogen, including effective amounts of the empathogen/entactogen and an adverse effect-reducing agent that blocks adverse effects of the empathogen/entactogen, such as reducing the acute cardiovascular stimulant and thermogenic effects of the empathogen/entactogen. The adverse effect-reducing agent is preferably carvedilol (FIG. 1B) or a similar adrenergic α/β receptor agonist or any combination of an α- and a β-blocker.

The empathogen/entactogen used to induce the desired psychological effects in the individual is preferably MDMA or an MDMA-like substance or derivative of MDMA or analog of MDMA with an MDMA-like empathogen/entactogen action.

While MDMA is referred to herein, it should be understood that any empathogen/entactogen or MDMA-like compound such as but not limited to 3,4-methylendioxyamphetamine (MDA), 3,4,-methylenedioxyethylamphetamine (MDEA), 5,6-methylenedioxy-2-aminoindane (MDAI), mephedrone, methylone, para-methamphetamine (PMA or PMMA), 3-methylmethcathinone (3-MMC), 4-fluoroamphetamine (4-FA), 2,5-dimethoxy-4-iodoamphetamine (DOI), 2,5-dimethoxy-4-bromoamphetamie (DOB), homologues thereof, analogues thereof, or novel compounds or prodrugs resulting in a similar MDMA-type acute subjective effect profile can be used in the methods herein. Any other compound that provides a similar MDMA-type acute subjective effect profile can also be used. In the methods herein, the empathogen/entactogen is preferably administered in a dose of 50-300 mg.

MDMA or MDMA-like substances are agonist agents that primarily release monoamines (serotonin, norepinephrine, and dopamine) and possibly also oxytocin typically by interacting with the membrane monoamine transporters (serotonin, norepinephrine, or dopamine transporter) (Hysek et al., 2014; Hysek et al., 2012b; Simmler et al., 2013; Verrico et al., 2007). This primary monoamine-releasing action of MDMA is thought to induce the psychological effects and it is not blocked by the blocking agent used in the present invention intended to block only adverse effects of MDMA. The blocking aging is used to prevent the released norepinephrine to interact with postsynaptic adrenergic α- and β-receptors and to thereby reduce the cardiovascular and thermogenic effects of MDMA.

While carvedilol is referred herein and used in the example study to illustrate the invention any other substance acting as a combined α- and β-receptor antagonist can be used as the adverse effect-reducing agent such as labetalol or others. Alternatively, two separate molecules, one blocking α-receptors and one blocking β-receptors can also be used in combination to generate the combined α- and β-receptor blockade. Examples of α-receptor blockers are prazosine or doxazosine, or any other blocker with a similar pharmacological action. Examples of β-receptor blockers are metoprolol, bisoprolol, atenolol, or any other blocker with a similar action. A substance blocking only α-receptors can also be used alone but would only be expected to be partly effective (Hysek et al., 2013a). The full benefits of the present invention can only be obtained by combined α- and β-receptor blockade (Hysek et al., 2012a). Using only β-receptor blockade with MDMA but without concomitantly blocking α-receptors is not advised due to a risk of increased blood pressure (Hysek et al., 2010). Among the β-receptor blockers to be used in the present invention, carvedilol is the preferred agent because it blocks receptors of the β3 subtype implicated in the thermogenic action of MDMA (Mills et al., 2003; Mills et al., 2004; Rusyniak et al., 2005; Sprague et al., 2004a; Sprague et al., 2004b; Sprague et al., 2005; Sprague et al., 2007). However, any other β-blocker which blocks also β3-subtype receptors can be equally suitable. Carvedilol can be administered in amounts of 5-150 mg.

Blockers of α-adrenergic receptors are widely used in medicine for the treatment of arterial hypertension and benign prostate hyperplasia. Blockers of β-adrenergic receptors are also widely used in the treatment of coronary artery disease and arterial hypertension and in patients with chronic heart failure.

Patients already treated with an α-blocker, a β-blocker, or a combination of the two would not need the addition of such blockers when treated with MDMA although an added benefit can still exist. In any case, any patient on a β-blocker receiving MDMA treatment should also be treated with a α-blocker or with carvedilol concomitantly. Accordingly, the present invention can also only use the combination of an α-blocker with the empathogen/entactogen in patients already on a β-blocker. An alternative preferred approach is to pause the β-blocker treatment on the day of the empathogen/entactogen administration and combine the empathogen/entactogen with an adverse effect-reducing agent such as carvedilol as proposed with the present invention.

In the present invention, the primary application provides a pharmaceutical combination. The combination product includes (i) an empathogen/entactogen inducing empathogenic subjective effects and (ii) an adverse effect-reducing agent of an adrenergic α-β-receptor antagonist which prevents, alleviates, and/or removes the cardiovascular stimulant and thermogenic effects of the empathogen/entactogen.

Within the present invention, the adverse effect-reducing agent and the empathogen/entactogen can also be used in combination but as separate dosage units. The adverse effect-reducing agent can be administered before or after the empathogen/entactogen, but administration together is preferred and the case when the empathogen/entactogen and the adverse effect-reducing agent are provided within the same composition.

As opposed to the prior art, the present invention uses the combined administration to prevent adverse effects of the empathogen/entactogen rather than treating adverse effects once established after empathogen/entactogen use. While the invention can be beneficial to reduce MDMA-associated adverse effects the approach herein is particularly useful in patients with an increased risk of cardiovascular complications such as patients with arterial hypertension, patients with coronary artery disease, and any patients were cardiovascular stimulation should be minimal or with an increased known or expected risk for hyperthermia.

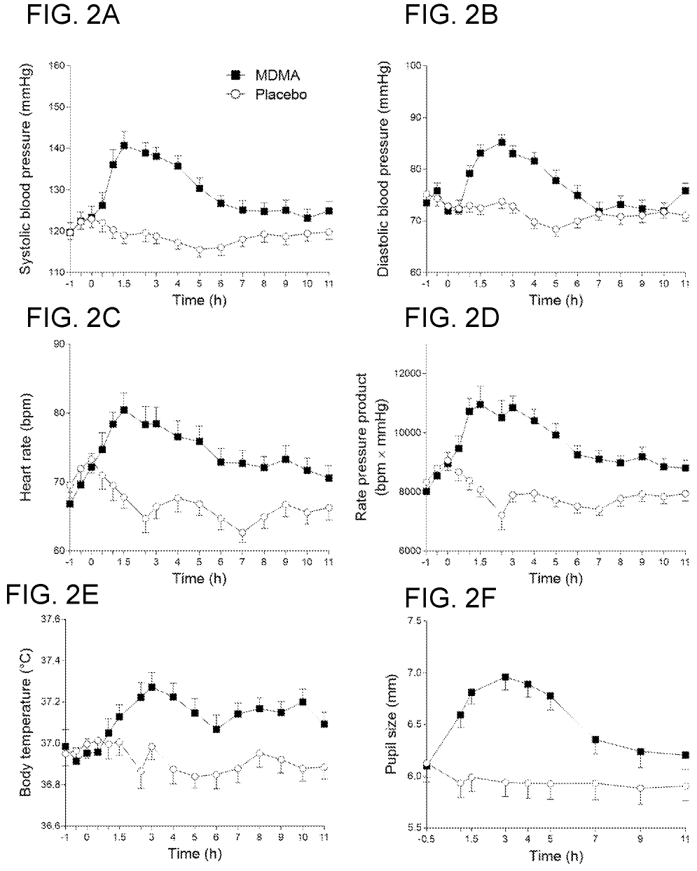

The sympathomimetic effects of MDMA are illustrated on FIGS. 2A-2F. The data is taken from a previously published study in the prior art (Holze et al., 2020) to illustrate the adverse effects of MDMA. In this randomized placebo-controlled study, 28 healthy human subjects were administered with MDMA (Holze et al., 2020) at a representative and commonly therapeutically used dose of 125 mg (Mithoefer et al., 2019) and placebo.

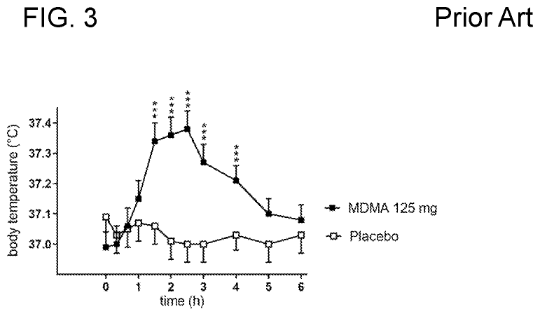

Blood pressure, heart rate, and tympanic body temperature were repeatedly measured 1 and 0.5 hour before and 0, 0.5, 1, 1.5, 2.5, 3, 4, 5, 6, 7, 8, 9, 10, and 11 hours after drug administration as previously described in detail (Hysek et al., 2010). Pupil function was measured under standardized dark-light conditions and assessed using a Voltcraft MS-1300 luxmeter (Voltcraft, Hirschau, Germany) after a dark adaption time of 1 minute as previously described (Hysek & Liechti, 2012). MDMA significantly increased the diastolic and systolic blood pressure, heart rate, body temperature, rate pressure product (heart rate×mean blood pressure product), and pupil size (Hysek & Liechti, 2012) (FIGS. 2A-2F). The acute effect of MDMA on the body temperature has also further been documented in a larger cohort of healthy subjects pooled form many randomized placebo-controlled studies (Liechti, 2014) and as illustrated in FIG. 3 (***for significant difference (p<0.001) vs. placebo). MDMA elevated body temperature even in non-ambulating persons acutely at 1.5-4 hours after administration compared with placebo administration in the same subjects.

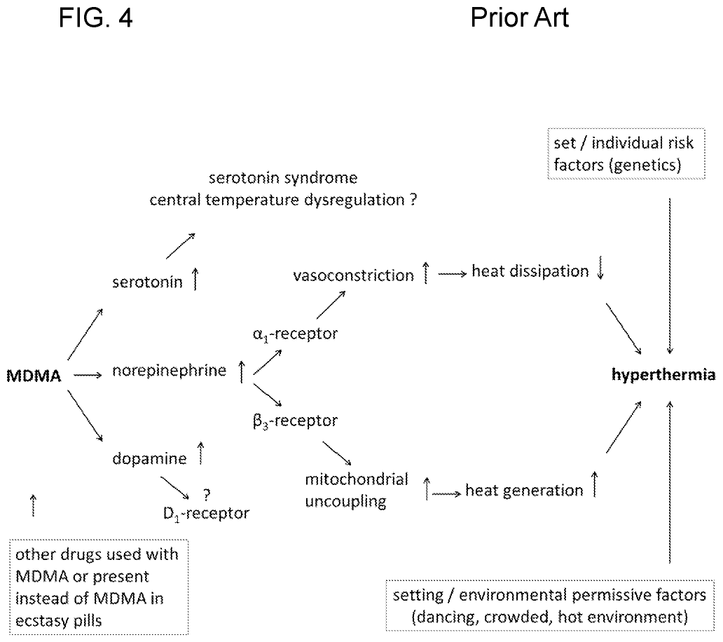

The mechanism by which MDMA is thought to induce body temperature is illustrated in FIG. 4 which has previously been published (Liechti, 2014). As can be seen in FIG. 4, effects of MDMA on body temperature are considered to depend on stimulation of adrenergic α1 and β3 receptors leading to both vasoconstriction and reduce heat dissipation (α1-mediated) and mitochondrial uncoupling and increased heat generation (β3-mediated). While this proposed mechanism has not been fully elucidated the concept of the present invention is blocking α1 and β3 receptors during the time of acute action of MDMA to prevent MDMA-induced heat generation and other unwanted sympathomimetic effects of MDMA while maintaining its emotional acute effects.

Several studies were conducted in healthy subjects to test the effect of adrenergic pretreatments on the thermogenic response to MDMA.

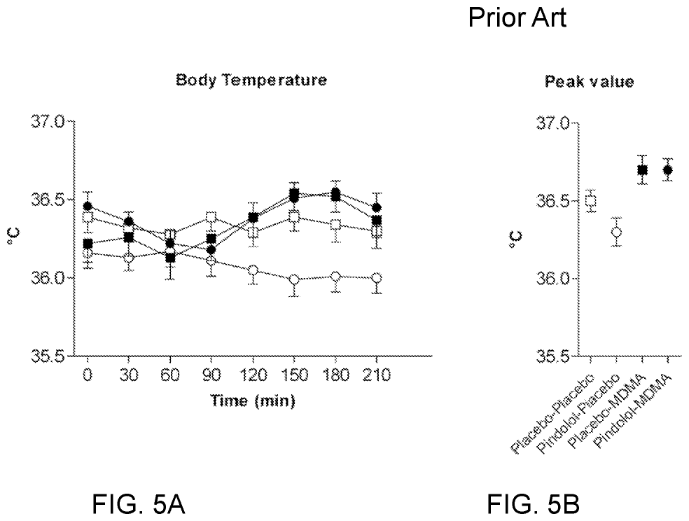

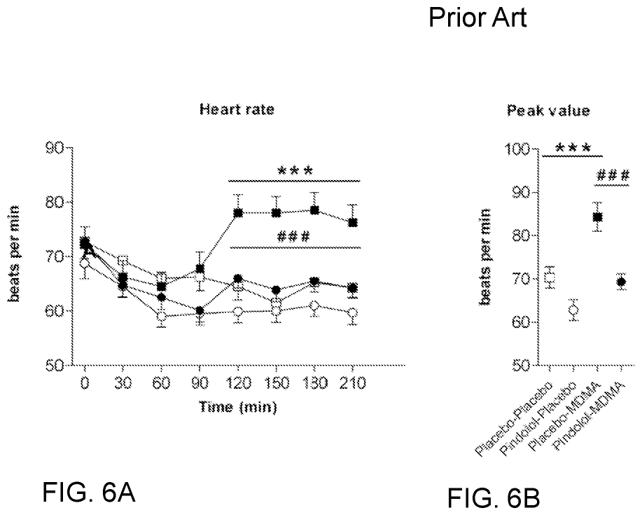

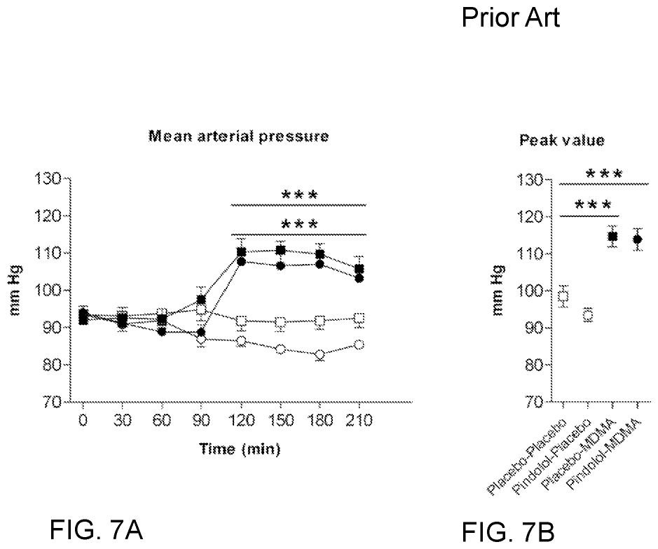

First, administration of a β1 receptor blocker (pindolol) had no effect on the MDMA-induced increase in body temperature in humans (FIGS. 5A-5B) (Hysek et al., 2010). Additionally, administration of the β1 receptor blocker pindolol reduced the MDMA-induced acute increase in heart rate (FIGS. 6A-6B) but not the increase in arterial blood pressure (FIGS. 7A-7B) (Hysek et al., 2010).

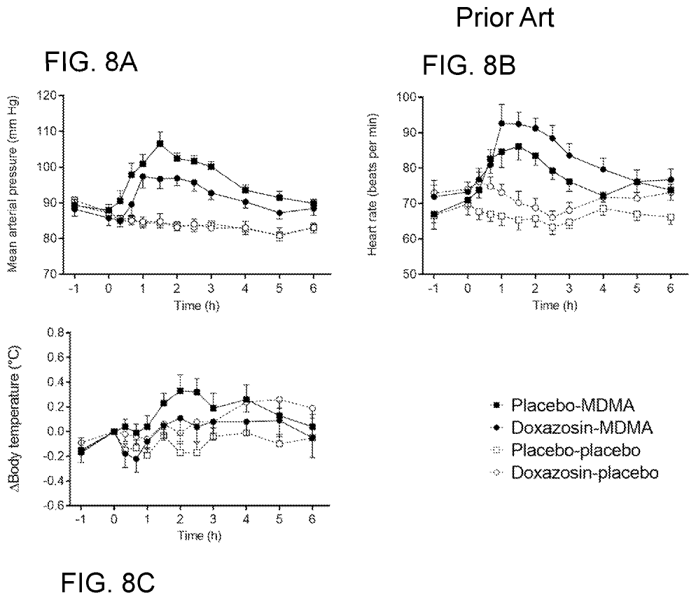

Second, another study tested the effect of pretreatment with an α1-adrenergic blocker (doxazosin) on the acute response to MDMA in healthy volunteers (Hysek et al., 2013a). Doxazosin reduced MDMA-induced elevations in blood pressure and body temperature but enhanced tachycardia associated with MDMA (FIGS. 8A-8C) (Hysek et al., 2013a). Thus, both α and β blockade were effective in reducing the response to MDMA but in differential and complementary ways reducing either blood pressure or heart rate while not affecting or even worsening the other. Therefore, the present invention made use of the combination of prior study findings.

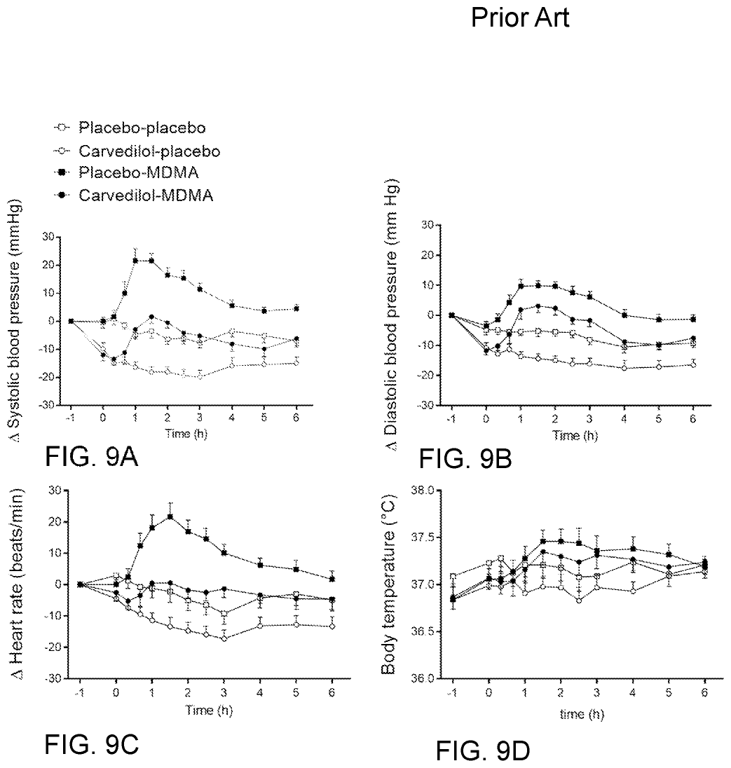

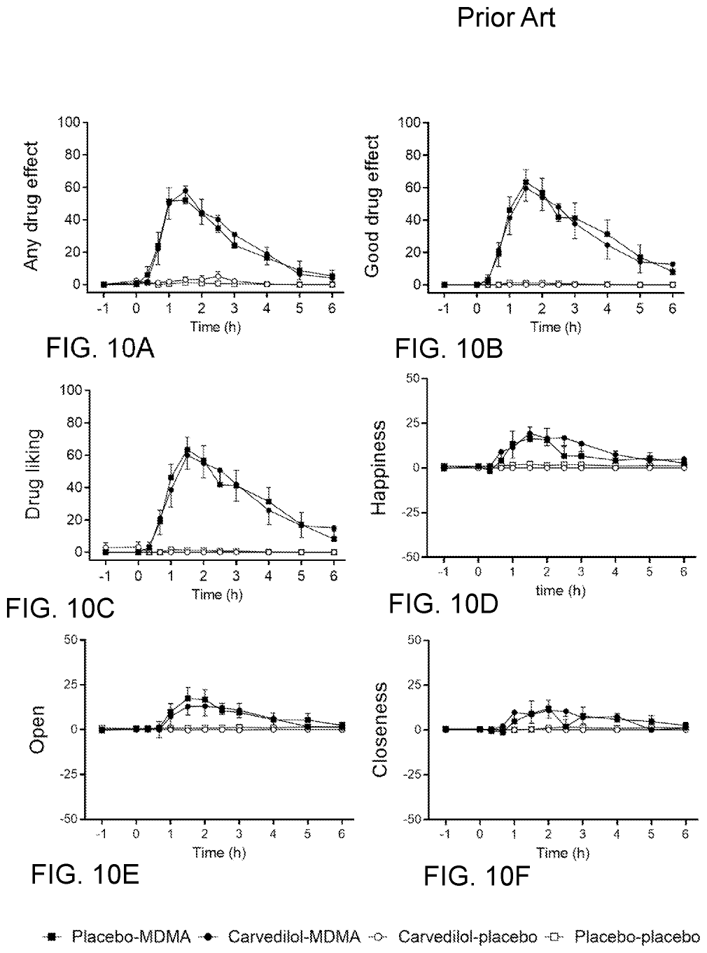

Furthermore, another study tested combined α1- and β-adrenergic blockade also including a wider range of β-receptor subtype blockade (namely β-3) based on preclinical data indicating efficacy of carvedilol to revert adverse sympathomimetic and thermogenic effects of MDMA (Sprague et al., 2005). This study showed that carvedilol administered before MDMA reduced MDMA-induced systolic and diastolic blood pressure as well as heart rate and body temperature (FIGS. 9A-9D). At the same time, carvedilol did not alter the desired subjective effects of MDMA (FIGS. 10A-10F).

The finding that carvedilol reduced the thermogenic effects of MDMA in both animals and humans provides critical support for the present invention. The present invention further aims to combine carvedilol and MDMA into one composition and co-administering the two rather than pretreating patients with carvedilol as was the concept in the past clinical proof of mechanism studies (Hysek et al., 2013b; Hysek et al., 2012a).

In the studies presented here, carvedilol was administered at a dose of 50 mg 1 hour before a standard therapeutic dose of 125 mg of MDMA in 16 healthy human subjects. The dose of 50 mg of carvedilol has previously been shown to attenuate the smoked cocaine-induced increases in heart rate and blood pressure in humans (Sofuoglu et al., 2000). At this dose, carvedilol is expected to inhibit both α1- and β-adrenoceptors (Sofuoglu et al., 2000; Tham et al., 1995). In this prior study, the maximal plasma concentration of carvedilol was expected to be reached before the maximal concentration of MDMA.

As a further development of this prior data, the present invention uses simultaneous administration of carvedilol with MDMA and ideally but not exclusively within the same composition. Additionally, the present invention is able to reduce the adverse effects of MDMA in all patients treated with MDMA or in patients at risk rather than treat only those cases with established side effects after using MDMA. Thus, carvedilol is administered with MDMA as a prophylactic to avoid or reduce its autonomic adverse effects while preserving its desired subjective effects.

Therefore, the present invention further developed the carvedilol-pre-dosing approach into a simultaneous dosing approach. The study provided the information on the effects of carvedilol alone, and MDMA alone. Further analyses of the data in FIGS. 9A-9D revealed that carvedilol and MDMA effects were additive regarding blood pressure and heart rate and interactive regarding body temperature.

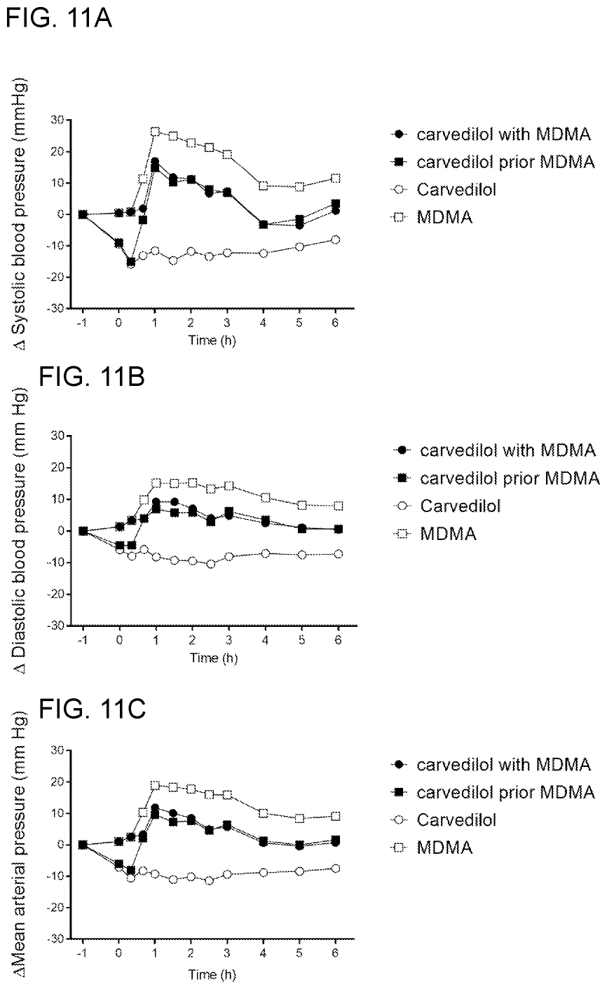

For blood pressure and heart rate the effects of carvedilol given simultaneously with MDMA were therefore estimated using the carvedilol and placebo mean values for each measured time point after administration and the mean carvedilol-placebo effect difference was calculated and then subtracted from the MDMA-placebo difference (FIGS. 11A-11C).

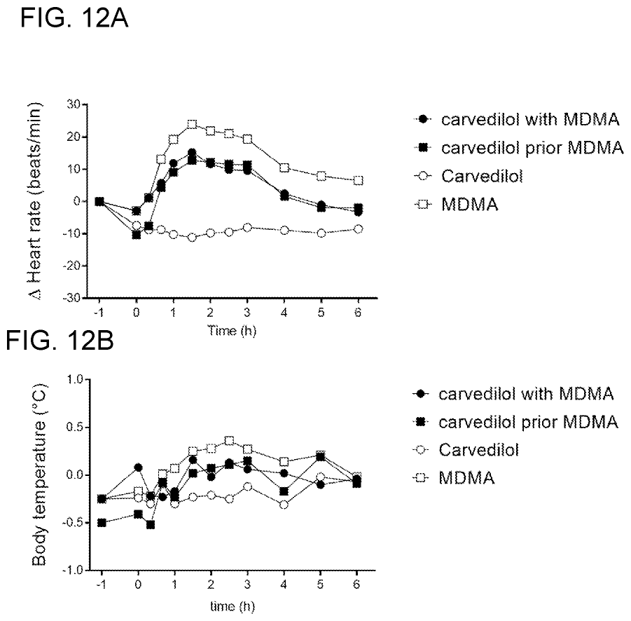

FIGS. 11A-11C and 12A-12B illustrate the beneficial effect of the present invention using co-administration of the adverse effect-reducing agent (carvedilol) and the empathogen/entactogen (MDMA). If carvedilol is administered before MDMA (carvedilol prior MDMA condition) as in the past study (Hysek et al., 2012a), a drop in blood pressure and heart rate can be observed in FIGS. 11A-11C and 12A-12B prior to the MDMA administration and consistent with the effect of carvedilol alone. While carvedilol pretreatment to MDMA effectively reduced the MDMA-induced increase in heart rate and blood pressure, administration of carvedilol with MDMA at the same time will not result in the drop prior to MDMA administration and reduce the MDMA effect effectively and similarly.

FIG. 12B shows that MDMA slightly increases body temperature. Carvedilol administered prior to MDMA and carvedilol administered with MDMA both reduce the MDMA-induced increase in body temperature (FIG. 12B)

The graphical illustrations (FIGS. 11A-11C and 12A-12B) are based on estimates and modeling using existing data from the pre-administration of carvedilol. However, it is clear from illustrations FIGS. 11A-11C and 12A-12B that the invention provides an effective approach to reduce MDMA-induced cardiostimulant effects. With regards to MDMA-induced increases in body temperature, the co-administration is expected to provide similar benefits to the pre-administration of carvedilol (FIGS. 9A-9D and 12A-12B) with a similar if not improved effect considering also that hyperthermia develops not instantly but often over hours after the MDMA administration and a shifted and later protection by carvedilol is likely even more beneficial.

The compounds used in the present invention are provided separately and administered orally. However, they can also be provided in the same dosage unit and have the same or different release profiles. For example, the dosage unit can be designed to release the empathogen/entactogen first and subsequently at a later time and using a different rate release the adverse effect-reducing agent. In another example, the empathogen/entactogen is released at a slow rate over 8 hours while the adverse effect-reducing agent is released more rapidly within 1-2 hours to be most effective when the peak effect of the empathogen/entactogen occurs after 1-2 hours after empathogen/entactogen administration.

The compound of the present invention is administered and dosed in accordance with good medical practice, taking into account the clinical condition of the individual patient, the site and method of administration, scheduling of administration, patient age, sex, body weight and other factors known to medical practitioners. For example, typical doses of MDMA are 75-200 mg with a preferred dose of 125 mg in men and 100 mg in women (Vizeli & Liechti, 2017).

The pharmaceutically “effective amount” for purposes herein is thus determined by such considerations as are known in the art (Mithoefer et al., 2019; Schmid et al., 2021; Studerus et al., 2021; Vizeli & Liechti, 2017). The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days in the case of the adverse effect-reducing agent. The treatment typically includes repeated single administrations of the empathogen/entactogen with typically several weeks between administrations and generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating an individual with an empathogen/entactogen while reducing its acute effects, by administering an empathogen/entactogen to the individual, administering an adverse effect-reducing agent such as carvedilol (or labetalol or similar) to the individual, and reducing the acute adverse effects of the empathogen/entactogen, while maintaining its psychotropic properties. This method can increase the safety profile of the empathogen/entactogen. The acute adverse effects that can be reduced include cardiostimulant and thermogenic effects. Any of the compounds described above can be administered and in amounts described above.

The present invention also provides for a method of stopping the acute cardiostimulant and hyperthermic action of an empathogen/entactogen in an individual, by administering an adverse effect-reducing agent such as carvedilol to the individual after the individual has taken the empathogen/entactogen and stopping the acute adverse effects of the empathogen/entactogen. As also described below, this method can be useful in stopping effects of the empathogen/entactogen that are having an adverse effect on an individual or in the case of an overdose. The adverse effect-reducing agent is efficacious in stopping acute effects of the empathogen/entactogen when administered after the empathogen/entactogen although the primary use of the present invention is to co-administer the adverse effect-reducing agent with the empathogen/entactogen at the same time. Any of the compounds described above can be administered and in amounts described above.

The invention allows MDMA side effects to be modified (attenuated) with the goal of reducing the acute stimulant and hyperthermic drug effect and/or duration with the goal of 1) reducing the cardiovascular and hyperthermia risk in any person compared to administering MDMA alone, 2) reducing the cardiotoxic risk in a patient at increased risk for cardiovascular events such as older patients or patients with coronary heart disease or arterial hypertension or any other disorder putting the patient at an increased risk for a hypertensive or cardiovascular adverse event, and 3) reducing the risk of hyperthermia in a patient with an increased risk for hyperthermia such as patients with a genetically higher risk such as malignant hyperthermia or in patients with hyperthermia in the past or in any cases where an MDMA-like substance is used with an known or suspected increased risk of hyperthermia including MDMA, para-methamphetamine (PMA or PMMA), 3-methylmethcathinone (3-MMC), 4-fluoroamphetamine (4-FA) or any similar serotonergic or stimulant or MDMA-like compounds (Luethi & Liechti, 2020).

Therefore, the present invention also provides for a method of treating an individual at risk of cardiovascular events and/or hyperthermia with an empathogen/entactogen, by administering an empathogen/entactogen to the individual, administering an adverse effect-reducing agent to the individual, and providing psychotropic effects of empathogen/entactogen to the individual while reducing risk of cardiovascular events and/or hyperthermia.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Dumont G J, Sweep F C, van der Steen R, Hermsen R, Donders A R, Touw D J, van Gerven J M, Buitelaar J K, & Verkes R J (2009). Increased oxytocin concentrations and prosocial feelings in humans after ecstasy (3,4-methylenedioxymethamphetamine) administration. Soc Neurosci 4: 359-366.
2. Green A R, O'Shea E, & Colado M I (2004). A review of the mechanisms involved in the acute MDMA (ecstasy)-induced hyperthermic response. Eur J Pharmacol 500: 3-13.
3. Hall A P, & Henry J A (2006). Acute toxic effects of ‘Ecstasy’ (MDMA) and related compounds: overview of pathophysiology and clinical management. Br J Anaesth 96: 678-685.
4. Holze F, Vizeli P, Muller F, Ley L, Duerig R, Varghese N, Eckert A, Borgwardt S, & Liechti M E (2020). Distinct acute effects of LSD, MDMA, and D-amphetamine in healthy subjects. Neuropsychopharmacology 45: 462-471.
5. Hysek C M, Fink A E, Simmler L D, Donzelli M, Grouzmann E, & Liechti M E (2013a). Alpha-adrenergic receptors contribute to the acute effects of MDMA in humans. J Clin Psychopharmacol 33: 658-666.
6. Hysek C M, & Liechti M E (2012). Effects of MDMA alone and after pretreatement with reboxetine, duloxetine, clonidine, carvedilol, and doxazosin on pupillary light reflex. Psychopharmacology 224: 363-376.
7. Hysek C M, Schmid Y, Rickli A, & Liechti M E (2013b). Carvedilol inhibits the cardiostimulant and thermogenic effects of MDMA in humans: lost in translation. Br J Pharmacol 170: 1273-1275.
8. Hysek C M, Schmid Y, Rickli A, Simmler L D, Donzelli M, Grouzmann E, & Liechti M E (2012a). Carvedilol inhibits the cardiostimulant and thermogenic effects of MDMA in humans. Br J Pharmacol 166: 2277-2288.
9. Hysek C M, Schmid Y, Simmler L D, Domes G, Heinrichs M, Eisenegger C, Preller K H, Quednow B B, & Liechti M E (2014). MDMA enhances emotional empathy and prosocial behavior. Soc Cogn Affect Neurosci 9: 1645-1652.
10. Hysek C M, Simmler L D, Nicola V, Vischer N, Donzelli M, KrahenbOhl S, Grouzmann E, Hoener M C, & Liechti M E (2012b). Duloxetine inhibits effects of MDMA (“ecstasy”) in vitro and in humans in a randomized placebo-controlled laboratory study. PLoS One 7: e36476.
11. Hysek C M, Vollenweider F X, & Liechti M E (2010). Effects of a b-blocker on the cardiovascular response to MDMA (ecstasy). Emerg Med J 27: 586-589.
12. Liechti M E (2014). Effects of MDMA on body temperature in humans. Temperature 1: 179-187.
13. Liechti M E, Baumann C, Gamma A, & Vollenweider F X (2000). Acute psychological effects of 3,4-methylenedioxymethamphetamine (MDMA, “Ecstasy”) are attenuated by the serotonin uptake inhibitor citalopram. Neuropsychopharmacology 22: 513-521.
14. Liechti M E, Kunz I, & Kupferschmidt H (2005). Acute medical problems due to Ecstasy use: case-series of emergency department visits. Swiss Med Wkly 135: 652-657.
15. Luethi D, & Liechti M E (2020). Designer drugs: mechanism of action and adverse effects. Arch Toxicol 94: 1085-1133.
16. Luoma J B, Chwyl C, Bathje G J, Davis A K, & Lancelotta R (2020). A Meta-Analysis of Placebo-Controlled Trials of Psychedelic-Assisted Therapy. J Psychoactive Drugs: 1-11.
17. Mills E M, Banks M L, Sprague J E, & Finkel T (2003). Pharmacology: uncoupling the agony from ecstasy. Nature 426: 403-404.
18. Mills E M, Rusyniak D E, & Sprague J E (2004). The role of the sympathetic nervous system and uncoupling proteins in the thermogenesis induced by 3,4-methylenedioxymethamphetamine. J Mol Med 82: 787-799.
19. Mithoefer M C, Feduccia A A, Jerome L, Mithoefer A, Wagner M, Walsh Z, Hamilton S, Yazar-Klosinski B, Emerson A, & Doblin R (2019). MDMA-assisted psychotherapy for treatment of PTSD: study design and rationale for phase 3 trials based on pooled analysis of six phase 2 randomized controlled trials. Psychopharmacology 236: 2735-2745.
20. Mithoefer M C, Wagner M T, Mithoefer A T, Jerome I, & Doblin R (2010). The safety and efficacy of ±3,4-methylenedioxymethamphetamine-assisted psychotherapy in subjects with chronic, treatment-resistant posttraumatic stress disorder: the first randomized controlled pilot study. J Psychopharmacol 25: 439-452.
21. Noseda R, Schmid Y, Scholz I, Liakoni E, Liechti M E, Dargan P I, Wood D M, Dines A M, Yates C, Heyerdahl F, Hovda K E, Giraudon I, & Ceschi A (2021). MDMA-related presentations to the emergency departments of the European Drug Emergencies Network plus (Euro-DEN Plus) over the four-year period 2014-2017. Clin Toxicol 59: 131-137.
22. Oehen P, Traber R, Widmer V, & Schnyder U (2013). A randomized, controlled pilot study of MDMA (±3,4-methylenedioxymethamphetamine)-assisted psychotherapy for treatment of resistant, chronic post-traumatic stress disorder (PTSD). J Psychopharmacol 27: 40-52.
23. Parrott A C (2012). MDMA and temperature: a review of the thermal effects of ‘Ecstasy’ in humans. Drug Alcohol Depend 121: 1-9.
24. Rosenson J, Smollin C, Sporer K A, Blanc P, & Olson K R (2007). Patterns of ecstasy-associated hyponatremia in California. Ann Emerg Med 49: 164-171.
25. Rusyniak D E, Tandy S L, Hekmatyar S K, Mills E, Smith D J, Bansal N, MacLellan D, Harper M E, & Sprague J E (2005). The role of mitochondrial uncoupling in 3,4-methylenedioxymethamphetamine-mediated skeletal muscle hyperthermia and rhabdomyolysis. J Pharmacol Exp Ther 313: 629-639.
26. Schmid Y, Gasser P, Oehen P, & Liechti M E (2021). Acute subjective effects in LSD- and MDMA-assisted psychotherapy. J Psychopharmacol. 35: 362-374
27. Schmid Y, Hysek C M, Simmler L D, Crockett M J, Quednow B B, & Liechti M E (2014). Differential effects of MDMA and methylphenidate on social cognition. J Psychopharmacol 28: 847-856.
28. Sessa B, Higbed L, & Nutt D (2019). A Review of 3,4-methylenedioxymethamphetamine (MDMA)-Assisted Psychotherapy. Front Psychiatry 10: 138.
29. Simmler L, Buser T, Donzelli M, Schramm Y, Dieu L H, Huwyler J, Chaboz S, Hoener M, & Liechti M E (2013). Pharmacological characterization of designer cathinones in vitro. Br J Pharmacol 168: 458-470.
30. Sofuoglu M, Brown S, Babb D A, Pentel P R, & Hatsukami D K (2000). Carvedilol affects the physiological and behavioral response to smoked cocaine in humans. Drug Alcohol Depend 60: 69-76.
31. Sprague J E, Banks M L, Cook V J, & Mills E M (2003). Hypothalamic-pituitary-thyroid axis and sympathetic nervous system involvement in hyperthermia induced by 3,4-methylenedioxymethamphetamine (Ecstasy). J Pharmacol Exp Ther 305: 159-166.
32. Sprague J E, Brutcher R E, Mills E M, Caden D, & Rusyniak D E (2004a). Attenuation of 3,4-methylenedioxymethamphetamine (MDMA, Ecstasy)-induced rhabdomyolysis with alpha1-plus beta3-adrenoreceptor antagonists. Br J Pharmacol 142: 667-670.
33. Sprague J E, Mallett N M, Rusyniak D E, & Mills E (2004b). UCP3 and thyroid hormone involvement in methamphetamine-induced hyperthermia. Biochem Pharmacol 68: 1339-1343.
34. Sprague J E, Moze P, Caden D, Rusyniak D E, Holmes C, Goldstein D S, & Mills E M (2005). Carvedilol reverses hyperthermia and attenuates rhabdomyolysis induced by 3,4-methylenedioxymethamphetamine (MDMA, Ecstasy) in an animal model. Crit Care Med 33: 1311-1316.
35. Sprague J E, Yang X, Sommers J, Gilman T L, & Mills E M (2007). Roles of norepinephrine, free Fatty acids, thyroid status, and skeletal muscle uncoupling protein 3 expression in sympathomimetic-induced thermogenesis. J Pharmacol Exp Ther 320: 274-280.
36. Studerus E, Vizeli P, Harder S, & Liechti M E (2021). Prediction of MDMA response in healthy humans: a pooled analysis of placebo-controlled studies. J Psychopharmacol 35: 556-565.
37. Tham T C, Guy S, McDermott B J, Shanks R G, & Riddell J G (1995). The dose dependency of the alpha- and beta-adrenoceptor antagonist activity of carvedilol in man. Br J Clin Pharmacol 40: 19-23.
38. Verrico C D, Miller G M, & Madras B K (2007). MDMA (ecstasy) and human dopamine, norepinephrine, and serotonin transporters: implications for MDMA-induced neurotoxicity and treatment. Psychopharmacology 189: 489-503.
39. Vizeli P, & Liechti M E (2017). Safety pharmacology of acute MDMA administration in healthy subjects. J Psychopharmacol 31: 576-588.
40. Vollenweider F X, Gamma A, Liechti M E, & Huber T (1998). Psychological and cardiovascular effects and short-term sequelae of MDMA (“ecstasy”) in MDMA-naive healthy volunteers. Neuropsychopharmacology 19: 241-251.

What is claimed is:

1. A composition for treating an individual while reducing acute adverse effects of an empathogen/entactogen, comprising effective amounts of an empathogen/entactogen and an adverse effect-reducing agent.

2. The composition of claim 1, wherein said empathogen/entactogen is a serotonin/oxytocin releaser chosen from the group consisting of MDMA, MDMA-analogs, MDMA-prodrugs, MDA, MDEA, MDAI, mephedrone, methylone, 3-MMC, 4-FA, PMA, PMMA, 2,5-dimethoxy-4-iodoamphetamine (DOI), 2,5-dimethoxy-4-bromoamphetamie (DOB), salts thereof, analogs thereof, and homologues thereof.

3. The composition of claim 1, wherein said empathogen/entactogen is present in an amount that results in cardiovascular stimulant and/or thermogenic effects.

4. The composition of claim 3, wherein said empathogen/entactogen is present in an amount of 50-300 mg and is chosen from the group consisting of MDMA, MDA, MDAI, mephedrone, methylone, or 3-MMC.

5. The composition of claim 1, wherein said adverse effect-reducing agent is an adrenergic α and β-receptor antagonist.

6. The composition of claim 5, wherein said adverse effect-reducing agent is chosen from the group consisting of carvedilol, labetalol, salts thereof, analogs thereof, and homologs thereof.

7. The composition of claim 6, wherein said carvedilol is present in an amount of 5-150 mg.

8. The composition of claim 1, wherein said empathogen/entactogen and adverse effect-reducing agent are in dosage units chosen from the group consisting of separate dosage units, in the same dosage unit with the same release profiles, and in the same dosage unit with different release profiles.

9. The composition of claim 1, wherein said adverse effect-reducing agent is an α-blocker.

10. A method of treating an individual with an empathogen/entactogen and reducing its acute adverse effects, including the steps of:
administering an empathogen/entactogen to the individual;
administering an adverse effect-reducing agent to the individual; and
reducing the acute adverse effects of the empathogen/entactogen.

11. The method of claim 10, wherein the adverse effect-reducing agent is administered at the same time or up to 24 hours after administering the empathogen/entactogen.

12. The method of claim 10, wherein the acute adverse effects are chosen from the group consisting of cardiostimulant effects and thermogenic effects.

13. The method of claim 10, wherein the empathogen/entactogen is a serotonin/oxytocin releaser chosen from the group consisting of MDMA, MDMA-analogs, MDMA-prodrugs, MDA, MDEA, MDAI, mephedrone, methylone, 3-MMC, 4-FA, PMA, PMMA, 2,5-dimethoxy-4-iodoamphetamine (DOI), 2,5-dimethoxy-4-bromoamphetamie (DOB), salts thereof, analogs thereof, and homologues thereof.

14. The method of claim 10, wherein the adverse effect-reducing agent is an adrenergic α and β-receptor antagonist.

15. The method of claim 10, wherein the adverse effect-reducing agent is chosen from the group consisting of carvedilol, labetalol, salts thereof, analogs thereof, and homologs thereof.

16. The method of claim 10, wherein the individual is already treated with a β-blocker and the adverse effect-reducing agent is an α-blocker.

17. A method of stopping acute cardiostimulant and hyperthermic actions of an empathogen/entactogen in an individual, including the steps of:
administering an adverse effect-reducing agent to the individual after the individual has taken the empathogen/entactogen; and
stopping the acute adverse effects of the empathogen/entactogen.

18. The method of claim 17, wherein the individual is experiencing adverse effects due to an overdose of the empathogen/entactogen.

19. The method of claim 17, wherein the empathogen/entactogen is a serotonin/oxytocin releaser chosen from the group consisting of MDMA, MDMA-analogs, MDMA-prodrugs, MDA, MDEA, MDAI, mephedrone, methylone, 3-MMC, 4-FA, PMA, PMMA, 2,5-dimethoxy-4-iodoamphetamine (DOI), 2,5-dimethoxy-4-bromoamphetamie (DOB), salts thereof, analogs thereof, and homologues thereof.

20. The method of claim 17, wherein the adverse effect-reducing agent is an adrenergic α and β-receptor antagonist.

21. The method of claim 17, wherein the adverse effect-reducing agent is chosen from the group consisting of carvedilol, labetalol, salts thereof, analogs thereof, and homologs thereof.

22. A method of treating an individual at risk of cardiovascular events and/or hyperthermia with an empathogen/entactogen, including the steps of:
administering an empathogen/entactogen to the individual who is at risk of cardiovascular events and/or hyperthermia;
administering an adverse effect-reducing agent to the individual; and
providing psychotropic effects of empathogen/entactogen to the individual while reducing risk of cardiovascular events and/or hyperthermia.

23. The method of claim 22, wherein the empathogen/entactogen is a serotonin/oxytocin releaser chosen from the group consisting of MDMA, MDMA-analogs, MDMA-prodrugs, MDA, MDEA, MDAI, mephedrone, methylone, 3-MMC, 4-FA, PMA, PMMA, 2,5-dimethoxy-4-iodoamphetamine (DOI), 2,5-dimethoxy-4-bromoamphetamie (DOB), salts thereof, analogs thereof, and homologues thereof.

24. The method of claim 22, wherein the adverse effect-reducing agent is an adrenergic α and β-receptor antagonist.

25. The method of claim 22, wherein the adverse effect-reducing agent is chosen from the group consisting of carvedilol, labetalol, salts thereof, analogs thereof, and homologs thereof.

26. The method of claim 22, wherein the individual is already treated with a β-blocker and the adverse effect-reducing agent is an α-blocker.
